(19) United States
(12) Patent Application Publication
(10) Pub. No.: US 2021/0069282 A1
(43) Pub. Date: Mar. 11, 2021

(54) NUTRITIONAL SUPPLEMENTS AFFECTING MOOD STATE AND SLEEP QUALITY

(71) Applicant: Amare Global, Draper, UT (US)

(72) Inventor: Shawn TALBOTT, Draper, UT (US)

(73) Assignee: Amare Global, Draper, UT (US)

(21) Appl. No.: 16/642,858

(22) PCT Filed: Aug. 30, 2018
(86) PCT No.: PCT/US2018/048945

§ 371 Date: Feb. 27, 2020

Related U.S. Application Data
(60) Provisional application No. 62/552,205, filed on Aug. 30, 2017.

(51) Int. Cl. A61K 36/899; A23L 33/105; A23L 33/155; A23L 33/16; A23L 33/00; A61K 33/06; A61K 33/30; A61K 31/375; A61K 31/593; A61K 31/714; A61K 31/4415; A61K 31/455; A61K 31/519; A61K 36/48; A61P 25/24; A61P 25/20

(52) U.S. Cl. CPC A61K 36/899; A23V 2002/00; A23L 33/155; A23L 33/16; A23L 33/40; A61K 33/06; A61K 33/30; A61K 31/375; A61K 31/593; A61K 31/714; A61K 31/4415; A61K 31/455; A61K 31/519; A61K 36/48; A61P 25/24; A61P 25/20; A23L 33/105

(57) ABSTRACT

Nutritional supplement compositions for enhancement of day-time mood and night-time sleep quality are disclosed. A nutritional supplement composition can include one or more of plant material from Corn grass, Griffonia seed, vitamin B3 (niacin), vitamin B6 (pyridoxine), vitamin B9 (folic acid), vitamin B12 (methylcobalamin), vitamin C (ascorbic acid), vitamin D (cholecalciferol), Magnesium, and Zinc. Related processes are also disclosed.

### TECHNICAL FIELD

The present disclosure relates generally to the field of nutritional treatments and supplements. More particularly, the present disclosure relates to nutritional treatments and supplements for, among other things, improving neurotransmitter metabolism, sleep quality, and one or more mood states of an individual.

### BACKGROUND OF THE INVENTION

Nutritional supplements are routinely used to improve health and/or physical performance. While nutritional supplements may be tailored to provide specific health and/or performance benefits, relatively few supplements provide such benefits while simultaneously improving both day-time mood state (depression/anxiety/vigor) and night-time sleep quality (improvement in both light stage REM sleep and deep stage restorative sleep).

### DETAILED DESCRIPTION

The present disclosure relates to nutritional treatments and supplements, and more particularly, to nutritional treatments and/or supplements that improve day-time mood state and night-time sleep quality of an individual. The following detailed description of various embodiments is not intended to limit the scope of the present disclosure, but is merely representative of various embodiments.

Amounts, concentrations, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, an amount of from 1 mg to 200 mg should be interpreted to include not only the explicitly recited limits of 1 mg and about 200 mg, but also to include individual amounts such as 2 mg, 3 mg, 4 mg, and sub-ranges such as 10 mg to 50 mg, 20 mg to 100 mg, etc. Unless otherwise stated, all ranges include both endpoints. The terms “negative mood state” and “positive mood state” are defined as described in Example 1 and elsewhere in the detailed description.

Nutritional supplements disclosed herein may include plant material from any combination of (1) Corn grass (Zea mays) and (2) Griffonia seed (Griffonia simplicifolia), plus (3) essential vitamin and mineral cofactors for serotonin/melatonin metabolism (Vitamins B3, B6, B9, B12, C, D, Magnesium, Zinc).

For example, in some embodiments, a nutritional supplement may include plant material from corn grass of the species Zea mays (commonly referred to as maize). In some embodiments, the plant material is extracted from the corn grass. More particularly, in some instances, the plant material is extracted to increase levels of 6-methoxybenzoxazolone (6-MBOA). In some instances, the nutritional supplement includes between about 10 and about 500 mg, between about 10 and about 75 mg; between about 10 and about 50 mg; between about 10 and about 25 mg; between about 25 and about 100 mg; between about 25 and about 75 mg; between about 25 and about 50 mg; or between about 35 and about 65 mg of plant material from corn grass, in some embodiments, from the species Zea mays. In some embodiments, plant material from Zea mays is between about 25% and about 75% of the nutritional supplement by weight.

In some embodiments, the species of the Griffonia seed from which plant material is derived may be Griffonia simplicifolia. More particularly, in some embodiments, the plant material, when dried, has a total 5-hyrdoxytryptophan (5-HTP) content of from about 50% to about 95% by weight. In some embodiments, the nutritional supplement includes between about 5 mg and about 200 mg, between about 25 mg and about 200 mg; between about 70 mg and about 200 mg; between about 100 mg and about 200 mg; between about 150 mg and about 200 mg; between about 15 mg and about 150 mg; between about 15 mg and about 100 mg; between about 15 mg and about 50 mg, between about 40 mg and about 130 mg; or between about 40 mg and about 75 mg of plant material from Griffonia seed. In some embodiments, plant material from Griffonia simplicifolia is between about 25% and about 75% of the nutritional supplement by weight.

In some embodiments, the combination of Zea mays and Griffonia seed are further combined with vitamin/mineral cofactors to support neurotransmitter metabolism. More particularly, these may include cofactors involved in metabolism of serotonin and melatonin. These, in turn, may include any combination of vitamin B3 (niacin), vitamin B6 (pyridoxine), vitamin B9 (folic acid), vitamin B12 (methylcobalamin), vitamin C (ascorbic acid), vitamin D (cholecalciferol), Magnesium, and Zinc.

In some embodiments, a nutritional supplement may be formulated as a capsule, tablet or softgel. For example, in some embodiments, ingredients of the nutritional supplement may be blended in various combinations described to form a single-dose delivery.

In some embodiments, a nutritional supplement may be formulated as a powdered drink mix. For example, in some embodiments, ingredients of the nutritional supplement may be mixed into a base liquid, such as water or juice to form a drinkable mixture or may be mixed into a base food, such as oatmeal or yogurt to form an edible mixture. In some embodiments, the liquid drink may be between about 1 and about 20 ounces in weight. In some embodiments, the nutritional supplement may be combined into a food base as a functional food including snack bars, cookies, wafers, and meal replacement shakes.

In some embodiments, the nutritional supplement may be effective for increasing positive mood state, as measured by self-reported feelings of energy, mood, focus, and well-being, when the nutritional supplement is administered to a healthy adult and/or a person suffering from mood disorders. For instance, in some embodiments, the nutritional supplement may increase average positive mood stateas measured on 100 mm visual analog scales (VAS)—by more than 10%, more than 25%, more than 40%, more than 60%, more than 70%, more than 80%, and/or more than 90%. Additionally or alternatively, average positive mood state may increase as a result of consumption of the nutritional supplement by more than approximately 10 VAS, 15 VAS, 20 VAS, 25 VAS, and/or 30 VAS, where each VAS unit corresponds to each mm along a 100 mm visual analog scale.

Additionally or alternatively, the nutritional supplement may, in some embodiments, be effective for decreasing negative mood state, as measured by self-reported feelings of stress, tension, irritability, and anxiety, when the nutritional supplement is administered to a healthy adult and/or a person suffering from mood disorders. In some embodiments, the nutritional supplement may decrease average negative mood stateas measured on 100 mm visual analog scales by more than 10%, more than 15%, more than 20%, more than 30%, more than 35%, and/or more than 40%. Additionally or alternatively, average negative mood state may decrease by more than 5 VAS, 8 VAS, 10 VAS, 12 VAS, and/or 15 VAS as a result of consumption of the nutritional supplement.

The nutritional supplement may, in some embodiments, be effective for improving time spent in REM (rapid eye movement) stages of sleep that are effective for brain rejuvenation benefits, and increase time spent in deep stages of “restorative” sleep that are effective for body recovery and restoration as measured by standardized sleep surveys or sleep monitors, when the nutritional supplement is administered to a healthy adult and/or a person suffering from sleep disorders.

### EXAMPLE 1

### Experimental Design

A series of studies were conducted to evaluate the effect of using a nutritional supplement combination that includes plant material from Corn grass (Zea mays) and Griffonia seed (Griffonia simplicifolia). More particularly, a multi-ingredient nutritional supplement that includes the ingredients set forth in Tables 1-2 was delivered orally to healthy adults. The amount of each listed ingredient in the nutritional supplement system is also set forth in the same table.

TABLE 1

| | Amount in a |
|---|---|
| Ingredient | single serving |
| Corn grass extract (Zea mays) and | 350 mg |
| Griffonia seed (Griffonia simplicifolia) | |

TABLE 2

| | Amount in a | |
|---|---|---|
| Ingredient | single serving | |
| Corn grass extract (Zea mays) and | 350 | mg |
| Griffonia seed (Griffonia simplicifolia) | | |
| Vitamin C | 22.5 | mg |
| Vitamin D | 150 | IU |
| Niacin | 4 | mg |
| Vitamin B6 | 425 | mcg |
| Folic Acid | 100 | mcg |
| Vitamin B12 | 1.5 | mcg |
| Magnesium | 50 | mg |
| Zinc | 2.8 | mg |

We supplemented moderately stressed subjects with a combination of Corn grass and Griffonia seed as described in Table 1 and Table 2.

Fifty-two subjects were randomly assigned in double-blind fashion to receive Corn grass (N=27, 18 Female & 9 Male) or Placebo (N=25, 17 Female & 8 Male) for 4 weeks.

We measured Mood State (Profile of Mood States), Sleep Quality (Pittsburgh Sleep Quality Index), and Sleep Patterns (ZEO Sleep Monitor) before and after 4 weeks of supplementation.

### RESULTS

Compared to the Placebo group, the Supplement group had the following results:
8% less Tension (7.9+5.9 v. 8.6+5.5)
15% less Depression (6.8+6.9 v. 8.0+7.9)
25% less Irritability (6.4+5.0 v. 8.0+7.9)
Fell asleep 33% faster (0.63+0.79 v. 0.84+0.90)
Woke up 30% fewer times each night (2.1+2.5 v. 3.0+1.5)
50% better sleep “efficiency” (0.26+0.59 v. 0.52+0.71)
40% better deep sleep “quality” (0.67+0.48 v. 1.12+0.97)
24% more time in deep REM sleep (1.85+0.46h v. 1.41+0.30h)

Overall, these results indicate that the Corn grass supplement is effective in improving sleep quality and improving stress-related mood states in a population of moderately stressed subjects. Future studies are warranted to evaluate the specific effects of MGE in alleviating over training syndrome (OTS) in athletes and possibly improving physical and mental performance.

In short, different levels of servings of the nutritional supplement described in this example unexpectedly produced both a decrease in negative mood state and an increase in positive mood state as well as an improvement in both light stage REM sleep (to support brain rejuvenation) and deep stage restorative sleep (to support body recovery and restoration).

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

This disclosure should not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

It will be apparent to those having skill in the art that changes may be made to the details of the above-described embodiments without departing from the underlying principles of the present disclosure.

1. A nutritional supplement comprising:
plant material from Corn grass; and plant material from Griffonia seed.

2. The nutritional supplement of claim 1, wherein the Corn grass is from the species Zea mays, and wherein the nutritional supplement comprises between about 25 mg and about 250 mg of corn grass extract.

3. The nutritional supplement of claim 1, wherein the Griffonia seed is from the species Griffonia simplicifolia, and wherein the nutritional supplement comprises comprising between about 10 mg and about 100 mg of Griffonia seed.

4. The nutritional supplement of claim 1, wherein the Corn grass and Griffonia seed are combined with vitamin/mineral cofactors for neurotransmitter metabolism (Vitamins B3, B6, B9, B12, C, D, Magnesium, Zinc).

5. The nutritional supplement of claim 1, wherein the nutritional supplement is effective for increasing positive mood state when administered to a healthy adult.

6. The nutritional supplement of claim 1, wherein the nutritional supplement is effective for decreasing negative mood state when administered to a healthy adult.

7. The nutritional supplement of claim 1, wherein the nutritional supplement is effective for improving sleep quality when administered to a healthy adult.

8. A method of altering the mood state of a subject, the method comprising:
obtaining the nutritional supplement of claim 1; and
ingesting the nutritional supplement.

9. A method of altering the mood state of a subject, the method comprising the steps of:
administering to the subject an effective amount of a nutritional supplement comprising plant material from Corn grass; and plant material from Griffonia seed.

10. The method of claim 9, wherein the plant material from Corn grass of the nutritional supplement is from the species Zea mays, and wherein the nutritional supplement includes between about 25 mg and about 250 mg of corn grass extract.

11. The method of claim 9, wherein the plant material from Griffonia seed is from the species Griffonia simplicifolia, and wherein the nutritional supplement includes between about 10 mg and about 100 mg of Griffonia seed.

12. The method of claim 9, wherein the plant material from Corn grass and the plant material from Griffonia seed are combined with vitamin/mineral cofactors for neurotransmitter metabolism (Vitamins B3, B6, B9, B12, C, D, Magnesium, Zinc).

13. The method of claim 9, wherein the method is effective for increasing positive mood state when administered to a healthy adult.

14. The method of claim 9, wherein the method is effective for decreasing negative mood state when administered to a healthy adult.

15. The method of claim 9, wherein the method is effective for improving sleep quality when administered to a healthy adult.

16. A method of altering the mood state of a human subject, the method comprising the steps of:
administering to the human subject an effective amount of a nutritional supplement comprising between about 25 mg and about 250 mg of corn grass extract from the species Zea mays; between about 10 mg and about 100 mg of Griffonia seed from the species Griffonia simplicifolia; and vitamin/mineral cofactors selected from the group consisting of Vitamin B3, Vitamin B6, Vitamin B9, Vitamin B12, Vitamin C, Vitamin D, Magnesium, and Zinc.

17. The method of claim 16, wherein the method is effective for increasing positive mood state when administered to a healthy adult.

18. The method of claim 16, wherein the method is effective for decreasing negative mood state when administered to a healthy adult.

19. The method of claim 16, wherein the method is effective for improving sleep quality when administered to a healthy adult.
